(19) United States
(12) Patent Application Publication
(10) Pub. No.: US 2009/0130176 A1
(43) Pub. Date: May 21, 2009

(54) INTRAOCULAR LENS WITH DRUG DELIVERY SYSTEM ATTACHED THERETO

(76) Inventors: Leila Bossy-Nobs, Geneve (CH); Olivia Felt-Baeyens, Feigeres (FR); Robert Gurny, Geneve (CH); Simone Eperon, Oron-Le-chatel (CH); Yan Guex-Crosier, La Conversion (CH)

Correspondence Address: BIRCH STEWART KOLASCH & BIRCH, PO BOX 747, FALLS CHURCH, VA, 22040-0747, US

(21) Appl. No.: 12/295,570

(22) PCT Filed: Mar. 29, 2007
(86) PCT No.: PCT/EP07/02832

§ 371 Date: Dec. 30, 2008

(30) Foreign Application Priority Data
Mar. 30, 2006 (EP) 06006803.8

(51) Int. Cl. A61K 9/00; A61P 27/02; A61K 31/58

(52) U.S. Cl. 424/428; 424/429; 514/174

(57) ABSTRACT

The present invention relates to a device comprising an intraocular lens and a drug delivery system attached thereto, wherein said drug delivery system is or can be loaded with one or more therapeutic agents. Preferably, said drug delivery system comprises a biodegradable matrix material wherein said biodegradable matrix material is preferably selected from the group consisting of: poly(lactic acid), poly(glycolic acid), poly(lactide-co-glycolide), polycaprolactones, polyorthoesters, polyanhydrides, polyesteramides and mixtures thereof. Preferred therapeutic agent(s) is/are selected from anti-inflammatory agents, anti-vasoproliferative agents, immunosuppressive agents, antibiotics, antiviral agents, anti-mitotic agents and combinations thereof. Also provided is a method of treatment and/or prevention of one or more diseases or malfunctions of the eye, comprising implanting one, two, three or more devices of the invention into an eye of a patient.

This invention relates to a device comprising an intraocular lens and a drug delivery system attached thereto, wherein said drug delivery system is or can be loaded with one or more therapeutic agents.

In this specification, a number of documents including patent applications and manufacturer's manuals is cited. The disclosure of these documents, while not considered relevant for the patentability of this invention, is herewith incorporated by reference in its entirety.

Each year, about two million people have cataract surgery and are implanted with an intraocular lens (IOL)¹. Intraocular inflammation is frequently observed following cataract extraction and can be controlled by the administration of anti-inflammatory drugs. In some cases, surgical trauma following cataract surgery can lead to cystoid macular edema (CME), one of the major cause of failure of ocular surgery². In the absence of specific anti-inflammation therapy, irreversible macular degeneration can lead to a permanent decrease in visual acuity³. Low-grade postoperative inflammation is also responsible for the proliferation of fibroblasts in the capsular bag and the development of opacification of the posterior capsule (secondary cataract). Moreover cataract surgery is performed mainly in elderly patients, who often suffer from combined ocular pathologies such as uveitis, macular degeneration or diabetic retinopathy. The presence of such pathologies is a major risk factor for the development of postoperative complications. These pathologies are worsened by the surgery and can lead to permanent visual loss. In the case of AMD for instance, ocular surgery may be followed by a rapid progression of choroidal neovascularization (CNV)¹⁸.

To control the intraocular inflammation following cataract surgery and in order to prevent short- and long-term complications, corticosteroids, such as triamcinolone acetonide (TA), and nonsteroidal anti-inflammatory drugs are applied after surgery. Simultaneous administration of anti-vasoproliferative agents (anti-VEGF) has also been suggested¹⁹.

However, it is difficult to deliver effective doses of drugs to the posterior part of the eye. Drugs can be applied either topically or systemically or by local injection. In the case of systemic administration large doses of the drug are needed to penetrate thought the blood-retinal barrier, resulting often in side effects⁴. Topical instillation of drugs has little therapeutic effect due to the poor penetration onto the posterior part of the eye. Intravitreous injection⁵ requires frequent injections in the vitreous to maintain the concentration of a drug within a therapeutic range over a long period of time and sometimes cause complications, such as vitreous haemorrhage, retinal detachment, or endophthalmitis⁶.

To overcome the drawbacks of these conventional treatments, a sustained-release drug delivery system may be the ideal solution to control postoperative inflammation following cataract surgery. The delivery system would be designed to increase the bioavailability, prolong controlled release of drugs, reduce systemic side effects and avoid repeated intraocular surgical procedures. Many solid drug delivery system offering sustained drug release into the posterior part of the eye have been extensively investigated⁷⁻¹⁰, however, only one biodegradable implant has been developed to treat inflammation following cataract surgery: the Surodex steroid DDS (Oculex Pharmaceuticals Sunnyvale, Calif.)^{11,12}. This device is placed into the eye at the conclusion of surgery. However, this device presents major disadvantages, such as migration of the device to anterior chamber, discomfort for the patients and the presence of residues of the devise after several months¹², thereby affecting visual acuity²⁰. Moreover, residues of implant can still be present after one year, as shown by gonioscopy. Obstruction of the angle may lead to severe ocular hypertension. The long discomfort was not negligible for patients. Finally, focal peripheral anterior synechiae lesions were observed in 22 eyes out of 71 eyes²⁰.

In view of the limitations of the—methods described in the prior art, the technical problem underlying the present invention was therefore the provision of alternative or improved means and methods for the treatment of eye diseases.

Accordingly, this invention relates to a device comprising an intraocular lens and a drug delivery system attached thereto, wherein said drug delivery system is or can be loaded with one or more therapeutic agents.

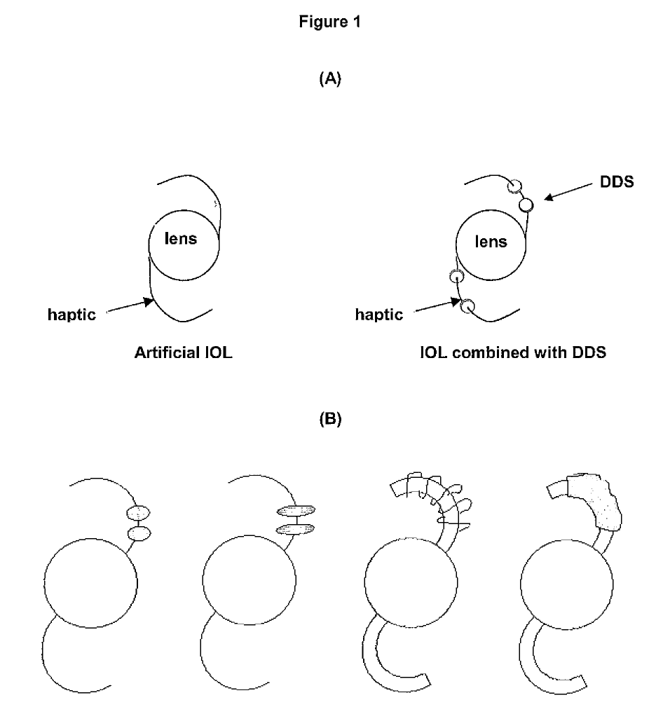

An intraocular lens (or IOL) according to the invention is a lens which is or is to be implanted in the eye. A schematic drawing of an IOL is provided in FIG. 1 (upper part, left). Usually an IOL replaces the existing crystalline lens, for example because it has been clouded over by a cataract. Alternatively, an intraocular lens may be implanted in addition to the existing crystalline lens. This type of IOL is also referred to as intraocular contact lens or implantable contact lens and is a small corrective lens that is surgically placed in the eye's posterior chamber behind the iris and in front of the lens to correct higher amounts of myopia and hyperopia.

Intraocular lenses usually consist of a plastic lens with plastic side struts called haptics to hold the lens in place within the capsular bag. Insertion of an intraocular lens is the most commonly performed eye surgical procedure; cataracts are the most common eye disease. The procedure can be done under local anaesthesia with the patient awake throughout the operation which usually takes less than 30 minutes in the hands of an experienced ophthalmologist. There are foldable intraocular lenses made of acrylic or silicone which can be rolled up and inserted through a tube with a very small incision not requiring any stitches; inflexible lenses (typically made of PMMA (polymethyl methacrylate)) require a larger incision.

The present invention may be used with intraocular lenses having haptics of any type. The haptics may be filiform (see FIGS. 1(A) and 4) or may be wider and/or thicker as shown in FIG. 1(B). Haptics of any further form or shape are also envisaged.

Unlike the natural lens, the curvature of current intraocular lenses typically can not be changed by the eye. Standard intraocular lenses provide good distance vision and the patient needs reading glasses for near vision. Newer bifocal intraocular lenses give distance vision in one area and near vision in another area of the vision field. There is also an FDA (Food and Drug Administration) approved lens called Crystalens whose position can be changed by the ciliary muscles of the eye, allowing for natural focusing. The drawback is the added expense of the lens and the need for a larger corneal incision for implantation.

A drug delivery system (DDS) according to the invention is a formulation or device that delivers drug(s) or therapeutic agent(s) to desired body location(s) and/or provides timely release of drug(s) or therapeutic agent(s). Desired body locations according to the invention include the intraocular region. The term “timely release” includes various release profiles well known in the art including prolonged release and/or delayed release. Drug delivery systems according to the invention preferably comprise a matrix material. The term “matrix material” relates to constituents of the DDS other than therapeutic agents. The one or more therapeutic agents are embedded or enclosed in said matrix material such that an immediate release of the entire amount of therapeutic agent is prevented.

The term “attached” refers to a physical connection between the intraocular lens and the drug delivery system. Preferably, the connection is such that routine handling of the device prior to implantation and wearing the device after implantation generally do not lead to a detachment of the drug delivery system from the intraocular lens. Furthermore, it is preferred that the drug delivery system is attached in such a way that vision is not impaired. Attachment may be such that a movement of the drug delivery system relative to the haptic is not possible. Alternatively, attachment may be such that the drug delivery system can be moved relative to the haptic of the intraocular lens. Preferred means of attaching are described further below.

Exemplary realizations of the device of the invention is shown in the photographs provided in FIG. 4. A schematic drawing is provided in FIG. 1 (upper part, right and bottom part). Accordingly, the present invention also relates to a device comprising an intraocular lens and one or more drug delivery systems attached thereto, wherein said drug delivery systems are or can be loaded with one or more therapeutic agents. Two, three of four drug delivery systems comprised in one device of the invention are deliberately envisaged.

In a preferred embodiment, more than one drug delivery system is attached to said intraocular lens, wherein different drug delivery systems are or can be loaded with different therapeutic agents. At the same time administration of a plurality of therapeutic agents may also be achieved by a device according to the invention comprising one or more drug delivery systems, wherein one or more of said drug delivery systems is or can be loaded with more than one therapeutic agent. Specifically, delivery of a plurality of therapeutic agents by using a single drug delivery device is envisaged. Accordingly, a plurality of drug delivery systems is not a prerequisite for administering a plurality of therapeutic agents, but may be employed for that purpose. On the other hand, in case only one therapeutic agent is to be delivered, it is nevertheless contemplated to use a plurality of drug delivery devices, for example to achieve delivery of a larger amount of drug, delivery of a larger dose per time unit, or a specific release characteristic. For example, the same therapeutic agent may be formulated into two different drug delivery devices which have different release characteristics, wherein at least one drug delivery device of either type is attached to the haptics of the intraocular lens. The skilled person can ensure a certain release characteristic by preparing a variety of drug delivery systems of different matrix material and/or under different conditions and testing them in in vitro and/or in vivo assays as described in the Examples enclosed herewith.

The terms “drug” and “therapeutic agent” are used equivalently herein. The term “comprising” as used herein is understood to include also “consisting of”.

The device of the invention permits to gather treatment of a disease requiring implantation of an intraocular lens (such as cataract) with the post-operative treatment in a single procedure. The device may be implanted in the eye with conventional surgical instruments as used in cataract surgery. The route of administration of therapeutic agents via a drug delivery system (DDS) attached to the intraocular lens increases the bioavailability and effectiveness of the drug as compared to the methods described in the art and reviewed herein above. The drug can be released at tailored and/or optimal concentrations over an extended period of time. As compared to routes of administration described in the art, the present invention permits the use of lower drug concentrations and/or dosages, thereby reducing side effects of the treatment. Patient non-compliance is prevented. Since the drug delivery device is attached to the intraocular lens, it is immobilized within the capsule. Migration of the device is thereby prevented.

Furthermore, the device of the invention permits implanting of a DDS during surgery, wherein the drug or one of the drugs to be released by the DDS may be directed to a further eye disease, i.e., a disease not or not directly linked to the condition to be ameliorated by the surgery or to possible conditions arising from the surgery. Indeed, patients having an intraocular lens implanted often suffer from other diseases such as uveitis, various retinopathies and age-related macular degeneration (AMD). Irrespective of whether these conditions may be worsened by operative intervention or not, they require additional treatments^{24,25}. As a consequence, implanting a device according to the invention during surgery not only facilitates post-operative treatments, but also minimizes the recurrence of patient pre-existing pathologies.

In a preferred embodiment said drug delivery system comprises a biodegradable matrix material. Biodegradability of the drug delivery system eliminates the need for a second surgery to remove the implanted drug delivery system. Preferably, the drug delivery system is biocompatible, i.e., it does not cause inflammation beyond the inflammation caused by the surgical intervention as such.

In a more preferred embodiment, said biodegradable matrix material is selected from the group consisting of: poly(lactic acid), poly(glycolic acid), poly(lactide-co-glycolide), polycaprolactones, polyorthoesters, polyanhydrides, polyesteramides and mixtures thereof. As is well known in the art, the polymers listed above can be prepared in different molecular weight ranges. The term “mixtures” also includes mixtures of polymers of the same type, but of different molecular weights. For example, a mixture of poly(lactic acid) with a molecular weight of about 10000 with poly(lactic acid) with a molecular weight of about 40000 may be used. In case of mixtures of polymers, one component may be present in an amount of 1%, 5%, 10%, 20%, 30%, 40%, 50%, 60%, 70%, 80%, 90%, 95% or 99%, wherein the percentages refer to weight percent.

When co-polymers are used as matrix materials, different blending ratios of the two or more building blocks of the co-polymer are deliberately envisaged. For example, poly(lactide-co-glycolide) can be prepared with different relative amounts of lactic acid and glycolic acid. For example, said relative amounts may be 10:90, 25:75, 50:50, 75:25 or 90:10. Co-polymers with different blending ratios may also be mixed with one another and/or with any other suitable matrix material including those explicitly disclosed herein.

Poly(lactide-co-glycolide) (PLGA) is a biodegradable polymer extensively used as to bioresorbable surgical sutures, implants, scaffolds for tissue-engineered skin and cartilage and various prosthetic devices¹³⁻¹⁵. As discussed below, there are various means of controlling the overall release profile of the drug from the drug delivery system according to the invention. The term “release profile” relates to the amount of drug release as a function of time. The term “overall release profile” relates to the release profile of the device according to the invention which is distinct from the release profile of a drug delivery system, in particular in those cases where the device according to the invention comprises more than one drug delivery system of different compositions.

As shown in the Examples enclosed herewith, the drug release profile can be controlled by varying the molecular weight of PLGA. Molecular weights in the range between 5000 and 500000 are deliberately envisaged as are ranges falling into this interval such as from 10000 to 100000. Particularly preferred is PLGA with a molecular weight between 30000 and 50000 such as 34000 or 48000. The respective lower values recited here are examples of “low molecular weight PLGA”, whereas the recited upper values are examples of “high molecular weight PLGA” according to the invention. Furthermore, different blending ratios of high and low molecular weight PLGA in one drug delivery system are deliberately envisaged, as are mixtures of poly(lactic acid) (PLA) and PLGA to prepare the drug delivery systems. For example, mixtures wherein the matrix comprises 1%, 5%, 10%, 20%, 30%, 40%, 50%, 60%, 70%, 80%, 90%, 95% or 99% low molecular weight PLGA may be used, wherein the remainder of the matrix consists of either high molecular weight PLGA or a mixture of high molecular weight PLGA and PLA. The percentages refer to weight percent.

The term “matrix material” designates the non-active agent component of the drug delivery system as opposed to the recited one or more therapeutic agents. Accordingly, in a preferred embodiment the drug delivery system consists of matrix material and one or more therapeutic agents.

Alternatively, further components or additives, for example components which modify the physical characteristics of the drug delivery system and/or its drug release characteristics may be present in small amounts, for example 10%, 5%, 2% or 1%. Said physical characteristics include the flexibility of the drug delivery system. In that case said additive is also referred to as a plastifier. Said drug release characteristics include the drug release rate. The drug release rate may be increased or decreased as compared to a drug delivery system without additives.

Alternatively or additionally, and in those cases where more than one drug delivery system is attached to the intraocular lens, one or more drug delivery systems comprising low molecular weight PLGA as matrix material and one or more drug delivery systems comprising high molecular weight PLGA as matrix material may be attached to the intraocular lens. The drug delivery system comprising low molecular weight PLGA as matrix material releases the drug completely or substantially within a period of weeks, for example one, two, three, four, five, six or seven weeks. The drug delivery system comprising high molecular weight PLGA as matrix material releases the drug over longer periods of time, for example, two, three, four, five or sixth months or periods up to one year.

Also, when more than one drug delivery system is attached to the intraocular lens, for each drug delivery system to be attached a chemically different matrix material may be used. For example, one DDS comprising PLGA as a matrix material and one DDS comprising PLA as matrix material may be attached to the same or different haptics of an intraocular lens to be implanted.

In a further preferred embodiment, said drug delivery system comprises a non-biodegradable matrix material.

In a more preferred embodiment, said non-biodegradable matrix material is selected from the group consisting of: methacrylates, acrylates and co-polymers thereof, silicone, and polymers and co-polymers of vinyl acetates, vinyl pyrrolidone and vinyl alcohol. Methacrylates of the invention include poly(methyl acrylate) (PMA) and poly(methyl methacrylate) (PMMA). Vinyl acetates include vinyl acetate and ethylene vinyl acetate (EVA).

Also envisaged are devices comprising one or more drug delivery systems, wherein a drug delivery system comprises a biodegradable and a non-biodegradable matrix material. Furthermore, devices are embraced wherein the matrix material of at least one drug delivery system is biodegradable and the matrix material of at least one drug delivery system is non-biodegradable.

In a further preferred embodiment, the release of said therapeutic agent(s) from said drug delivery system occurs within a period of up to one year. The term “release occurs within a period of up to one year” means that 50%, 60%, 70%, 80%, 900%, 95, 99% or 100% of the therapeutic agent(s) is/are released from the drug delivery system within a period of one, two, three, four, five, six, seven, eight, nine or ten weeks, or three, four, five, six, seven, eight, nine, ten or eleven months or one year or more than one year. Particularly preferred is a release of about 40 to 50 weight percent within a period of about six weeks. Also preferred is a linear release, i.e., the amount of therapeutic agent release per time unit is constant.

In a further preferred embodiment, said drug delivery system is attached to a haptic of said intraocular lens.

The term “haptic” designates the elongated structure attached to the lens part of an intraocular lens. The haptics permit placement and immobilization of the intraocular lens. As shown in FIGS. 1 and 4, intraocular lenses preferably have two haptics. One or more drug delivery systems may be attached to each haptic. As a consequence, a device of the invention may carry one, two, three, four or more drug delivery systems.

Preferably the shape of said drug delivery system is selected from: disc, cylinder, nail-like-plug, rod, tablet, sphere, truncated cone with a bended axis, polymer wire rolled around a haptic, and polymer sheet wrapped around a haptic. In case of a disc-shaped drug delivery system, the attachment of the drug delivery system to a haptic of the intraocular lens may be achieved by a small hole in the disc, for example in the center of the disc, wherein the size of the hole is such that the haptic may be inserted into the hole (see FIG. 4). Such a drug delivery device is particularly suitable for filiform haptics, but not limited thereto. As indicated in FIG. 1 (upper part, right and bottom part), the attachment of more than one drug delivery system may easily be achieved by these means. Polymer wires and polymer sheets may be sealed by heating to ensure that the drug delivery device remains attached to the haptic. Polymer sheets are particularly suitable for lenses with wider or thicker haptics such as one-piece lenses, but may also be used for attachment to filiform haptics.

In a preferred embodiment, said therapeutic agent(s) is/are selected from anti-inflammatory agents, anti-vasoproliferative agents, immunosuppressive agents, antibiotics, antiviral agents, anti-mitotic agents and combinations thereof.

Preferably, said anti-inflammatory agents are selected from the group consisting of corticosteroids, cycloplegics and non-steroidal anti-inflammatory drugs.

In a more preferred embodiment, said therapeutic agent(s) is/are selected from one or more of the following: triamcinolone acetonide, betamethasone, dexamethasone, prednisolone, fluorometholone, anti-VEGF agents such as the agents described in reference 19, anti-TNF agents, ganciclovir, acyclovir and foscavir.

Triamcinolone acetonide (TA) is a particularly preferred anti-inflammatory agent to be used in a device of the invention. Examples 4 and 5 enclosed herewith and the Figures referenced therein provide in vivo data obtained with a device according to the invention loaded with TA.

Anti-VEGF agents include aptamers, antisense oligonucleotides and anti-VEGF antibodies, preferably anti-VEGF monoclonal antibodies²⁹. An example of an anti-VEGF aptamer is Pegaptanib sodium which is a pegylated anti-VEGF aptamer³⁰. A recombinant humanized monoclonal antibody directed towards VEGF is described in reference 31. A further example is the monoclonal antibody bevacizumab (Avastin, Genentech, South San Francisco, Calif.)³². AS-ODN³³ is an example of an antisense oligonucleotide directed against VEGF.

Anti-TNF agents according to the invention include infliximab, a mouse-human chimeric immunoglobulin G1 monoclonal antibody³⁴, etanercept which is a competitive inhibitor of TNF³⁴, adalimumab and related drugs.

In a further preferred embodiment, said therapeutic agent(s) is/are selected from the group consisting of: antisense oligonucleotides, aptamers and antibodies and fragments thereof. Said agents are directed to target molecules whose inhibition or activation, respectively, is beneficial to the condition to be treated. The term “target molecule” designates a naturally occurring biomolecule which is disease-relevant in a given patient. Preferred target molecules include VEGF and TNF (see above).

Further preferred therapeutic agents include peptides and constructs for gene therapy. Constructs for gene therapy are well known in the art and familiar to the skilled person. Constructs for gene therapy generally comprise (i) a therapeutic agent which is generally a nucleic acid and which is to be delivered to a target site, target cell and/or target tissue and (ii) an agent which enables of facilitates delivery to said target site, target cell and/or target tissue such that the therapeutic effect may occur or is enhanced. For example, the nucleic acid to be delivered may be comprised in a vector, preferably a viral vector. Viral vectors for gene therapy include retroviruses, adenoviruses, adeno-associated viruses. Furthermore, many vectors have been developed in which the endogenous viral envelope proteins have been replaced by either envelope proteins from other viruses, or by chimeric proteins. Such chimera consist of those parts of the viral protein necessary for incorporation into the virion as well as sequences meant to interact with specific host cell proteins. Viruses in which the envelope proteins have been replaced as described are referred to as pseudotyped viruses. The above described procedure is also referred to as envelope protein pseudotyping of viral vectors.

Another type of constructs for gene therapy are lipoplexes and polyplexes. A lipoplex is a liposome complexed with nucleic acid. Polyplexes are complexes of polymers, preferably cationic polymers with nucleic acid. Lipoplexes and polyplexes protect the nucleic acid from undesirable degradation during the transfection process. Under certain conditions component (i) alone may suffice and constitute the construct for gene therapy according to the invention. Example are naked DNA and oligonucleotides. Oligonucleotides for gene therapy include antisense oligonucleotides and siRNAs.

The term “antibody” includes monoclonal antibodies, polyclonal antibodies, single chain antibodies, or fragments thereof that specifically bind said polypeptide, also including bispecific antibodies, synthetic antibodies, antibody fragments, such as Fab, a F(ab₂)′, Fv or scFv fragments etc., or a chemically modified derivative of any of these. Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals with modifications developed by the art. Furthermore, antibodies or fragments thereof to the aforementioned polypeptides can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to an epitope (Schier, Human Antibodies Hybridomas 7 (1996), 97-105; Malmborg, J. Immunol. Methods 183 (1995), 7-13). The production of chimeric antibodies is described, for example, in WO89/09622. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735. Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook (1989), loc. cit.

The term “monoclonal” or “polyclonal antibody” (see Harlow and Lane, (1988), loc. cit.) also relates to derivatives of said antibodies which retain or essentially retain their binding specificity. Preferred derivatives of such antibodies are chimeric antibodies comprising, for example, a mouse or rat variable region and a human constant region.

The term “scFv fragment” (single-chain Fv fragment) is well understood in the art and preferred due to its small size and the possibility to recombinantly produce such fragments.

The term “specifically binds” in connection with the antibody used in accordance with the present invention means that the antibody etc. does not or essentially does not cross-react with (poly)peptides of similar structures. Cross-reactivity of a panel of antibodies etc. under investigation may be tested, for example, by assessing binding of said panel of antibodies etc. under conventional conditions (see, e.g., Harlow and Lane, (1988), loc. cit.) to the (poly)peptide of interest as well as to a number of more or less (structurally and/or functionally) closely related (poly)peptides. Only those antibodies that bind to the (poly)peptide/protein of interest but do not or do not essentially bind to any of the other (poly)peptides which are preferably expressed by the same tissue as the (poly)peptide of interest, are considered specific for the (poly)peptide/protein of interest.

In a particularly preferred embodiment of the method of the invention, said antibody or antibody binding portion is or is derived from a human antibody or a humanized antibody.

The term “humanized antibody” means, in accordance with the present invention, an antibody of non-human origin, where at least one complementarity determining region (CDR) in the variable regions such as the CDR3 and preferably all 6 CDRs have been replaced by CDRs of an antibody of human origin having a desired specificity. Optionally, the non-human constant region(s) of the antibody has/have been replaced by (a) constant region(s) of a human antibody. Methods for the production of humanized antibodies are described in, e.g., EP-A1 0 239 400 and WO90/07861.

Said therapeutic agent(s) may furthermore be selected from molecules which activate or inhibit gene expression. Said molecules may act at the level of transcription by interfering with RNA production and/or may act at the level of translation, i.e., by interfering with the production of protein. Further envisaged therapeutic agents are transporters which are capable of carrying transcription factors, inhibitory domains and/or activating domains.

The present invention also relates to the use of a an intraocular lens and a drug delivery system attached thereto, wherein said drug delivery system is or can be loaded with one or more therapeutic agents, for the manufacture of a device for the treatment and/or prevention of one or more diseases or malfunctions of the eye. The device to be manufactured may be a device as defined in any one of the embodiments disclosed herein above. Also provided is the use of a an intraocular lens and one or more drug delivery systems, wherein said drug delivery systems are or can be loaded with one or more therapeutic agents, for the manufacture of a device for the treatment and/or prevention of one or more diseases or malfunctions of the eye, wherein during the course of the manufacture said drug delivery systems are attached to said intraocular lens.

Also provided is a method of treatment and/or prevention of one or more diseases or malfunctions of the eye, comprising implanting a device of the invention into an eye of a patient.

In a preferred embodiment of the method of the invention, said implanting is effected by using conventional surgical instruments as used in cataract surgery. In other words, the step of implanting does not pose requirements different from those in normal eye surgery.

In a preferred embodiment of the method or the use of the invention, said disease or malfunction is selected from the group consisting of: a condition requiring implantation of an intraocular lens, a condition arising from or aggravated by the implantation of an intraocular lens, a condition present before implantation, and combinations thereof.

The skilled person is aware of conditions requiring implantation of an intraocular lens. Said conditions comprise all forms of impaired vision, wherein the vision impairment results from deficiencies of the lens. Lens deficiencies include lens opacification and improper lens refractivity.

The term “condition arising from or aggravated by the implantation of an intraocular lens” designates post-operative complications which may occur as a consequence of eye surgery. The skilled person is aware of all forms of complications which may possibly occur. Exemplary complications are described herein above and further detailed below.

The term “condition present before implantation” relates to conditions which are not or not directly linked to the conditions being treated by surgery (such as cataract) nor to any of the post-operative complications. Rather, it relates to disease which may be concomitantly treated with the surgical intervention.

In a preferred embodiment of the method or the use of the invention, said condition requiring implantation of an intraocular lens is cataract or is expected to result in cataract. In opthalmology, a cataract is any opacity which develops in the crystalline lens of the eye or in its envelope. Cataracts form for a variety of reasons, including long-term ultraviolet exposure, secondary effects of diseases such as diabetes, or simply due to advanced age; they are usually a result of denaturation of lens proteins. Genetic factors are often a cause of congenital cataracts and may also play a role in predisposing someone to cataracts. Some cataract formation is to be expected in any person over the age of 70. There are different forms of cataract. Cataract may be classified by etiology. Accordingly, the term “cataract” as used herein includes age-related cataract, congenital cataract, secondary cataract and traumatic cataract. Alternatively or additionally, cataract may be classified by location. Accordingly, the term “cataract” as used herein includes anterior cortical cataract, anterior polar cataract, anterior subcapsular cataract, nuclear cataract, posterior cortical cataract, posterior polar cataract and posterior subcapsular cataract.

Additional or alternative conditions requiring implantation of an intraocular lens include myopia, hyperopia and astigmatism. In these cases the preferred type of IOL is an intraocular contact lens as defined above.

In a preferred embodiment of said use or said method, said condition arising from or aggravated by the implantation of an intraocular lens is selected from: postoperative inflammation, capsule opacification (secondary cataract), uveitis, cystoid macular edema (CME) and retinopathies.

In another preferred embodiment of the use or the method of the invention, said disease or malfunction is selected from the group consisting of: uveitis; age-related macular degeneration (AMD); retinopathies including diabetic retinopathy, retinitis including retinitis pigmentosa, viral retinitis such as cytomegalovirus (CMV) retinitis, and proliferative vitreoretinopathy; endophthalmitis and choroidal neovascularization.

To date, AMD represents the most common source of blindness in elderly people of the developed countries. Among these patients, about 15% show the wet form, leading to choroidal neovascularization^{22,26}. This chronic disease has restrained improvement in visual acuity, despite a variety of treatments like photodynamic therapy and laser photocoagulation²⁸. Although macular translocation improves the visual acuity, it is often associated with severe complications. Anti-angiogenic agents like anti-VEGF has been demonstrated to greatly inhibit neovascularization²⁷. Up to now, the difficulty was to maintain the active agent in effective concentrations. The implantation of a device of the invention comprising a DDS comprising an anti-VEGF permits slow delivery over a prolonged period of time.

The Figures show:

FIG. 1: (A) A schematic representation of the IOL combined with drug delivery system (DDS). Up to four (see schematic) or more DDS can be attached to the IOL. (B) further exemplary embodiments of IOLs combined with DDS: discs or spheric tablets threaded onto the haptic; ellipsoid tablets threaded onto the haptic; polymer wire rolled around the haptic; polymer sheet wrapped around the haptic (from left to right). The first two drawings represent IOLs with filiform haptics, the two drawings on the righthand side represent one-piece lenses with wider haptics.

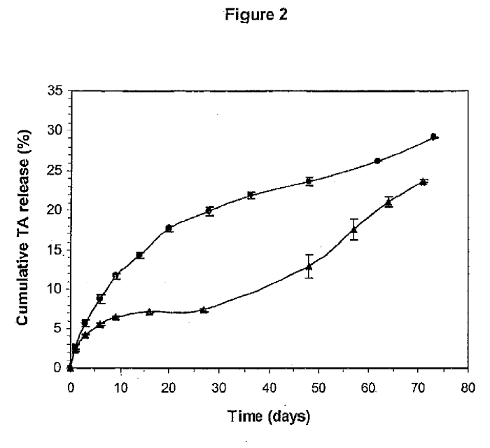

FIG. 2: Effects of the molecular weight of PLGA on in the vitro release from implants at constant TA loading of 35%. • implants PLGA-34000, ▴ implants PLGA-48000. The values are shown as mean±SD of n=6.

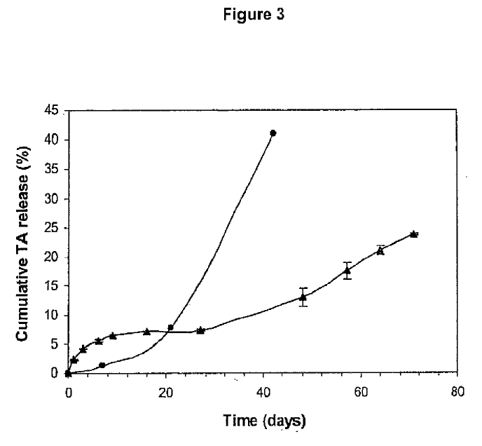

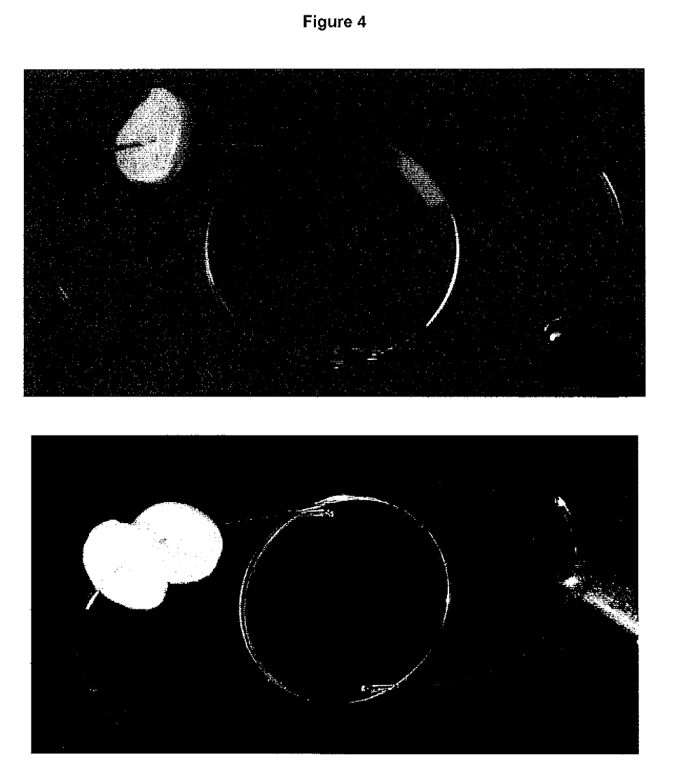

FIG. 3: In vivo and in vitro release profile of TA from the intraocular implants. ▴ in vitro implants (PLGA48000b), • in vivo implants (PLGA48000b). The data are the mean±SD of results in 6 experiments for in vitro implants and mean of 2 experiments for in vivo implants.

FIG. 4: The IOLs are made of an optic center and 2 haptics. The DDS is a disc with a hole in its center. One or two DDS are stringed on one haptic of the IOL. The DDS-IOLs are handled by forceps to take the picture.

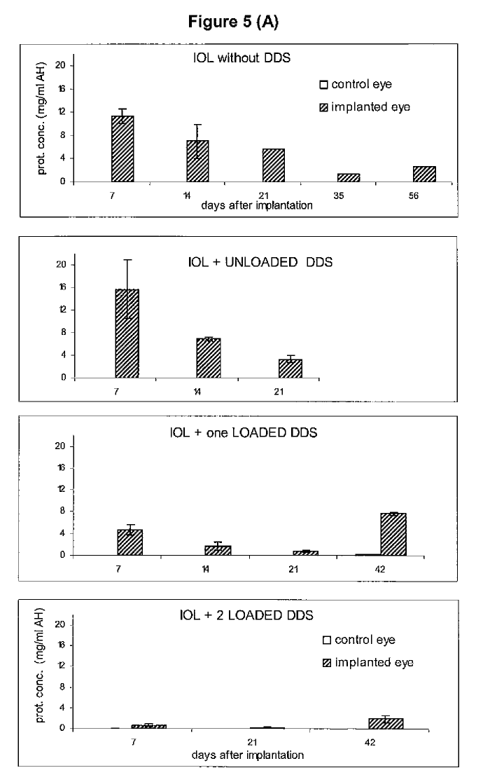

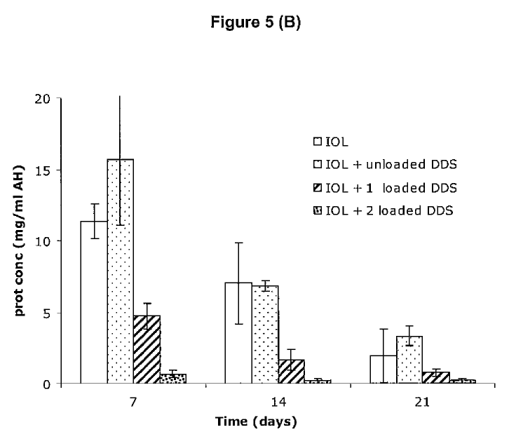

FIG. 5: (A) Protein concentration in mg/ml aqueous humor (AH)±SEM after cataract surgery. Insertion of IOL without DDS (n=2), IOL+unloaded DDS (n=4), IOL+one loaded DDS (n=5), IOL+two loaded DDS (n=2). (B) Further data have been added and displayed differently.

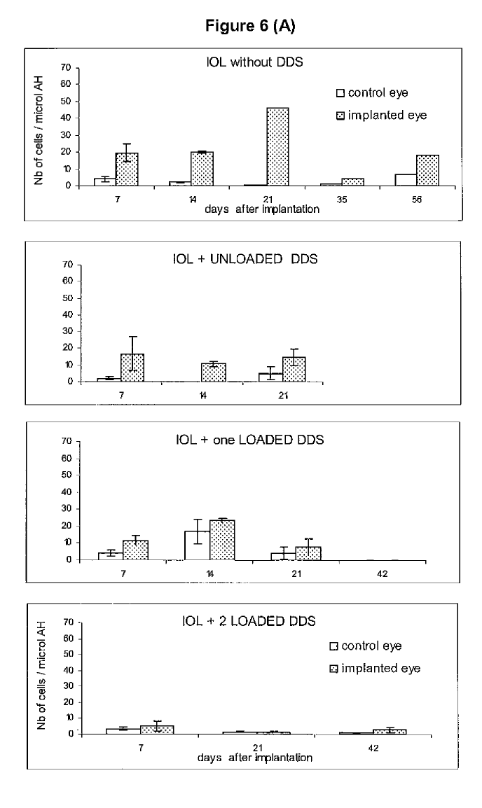

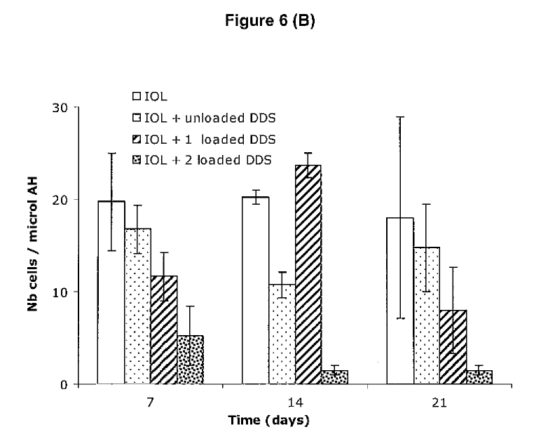

FIG. 6: (A) Number of cells/microlitre AH±SEM after cataract surgery. Same legend as in previous figure. (B) Further data have been added and displayed differently.

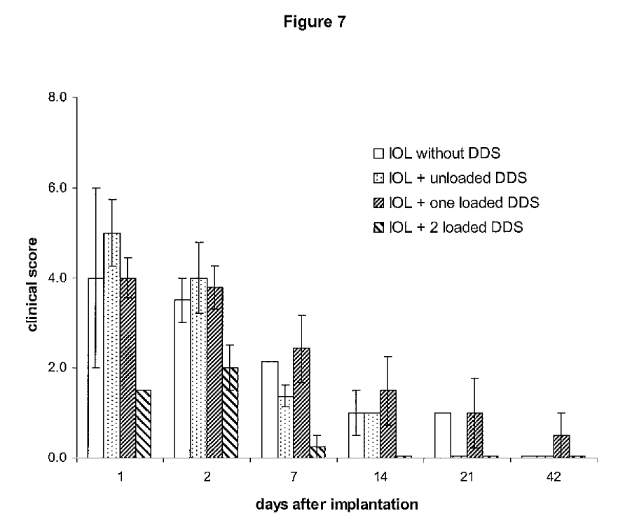

FIG. 7: Clinical score of the eye implanted with IOL±SEM after cataract surgery. The clinical score of the control eye was always equal to zero. Same legend as in previous figure.

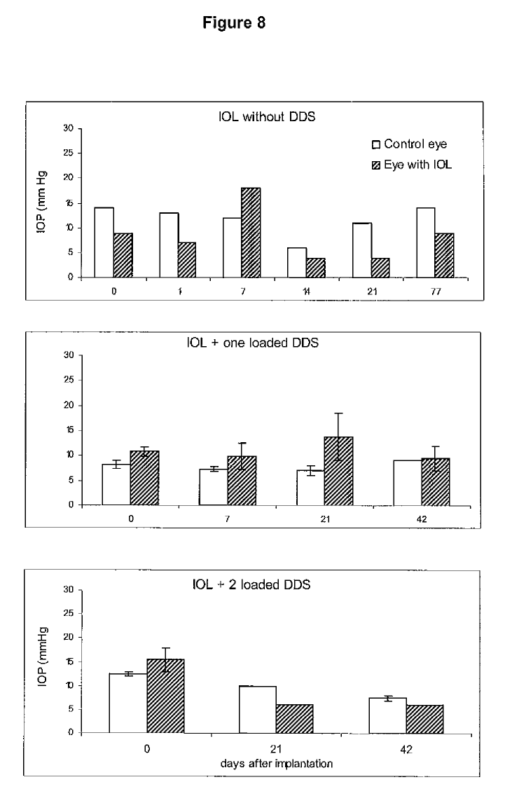

FIG. 8: Intraocular pressure (IOP) in mm Hg in control eye and in eye implanted with IOL±SEM after cataract surgery.

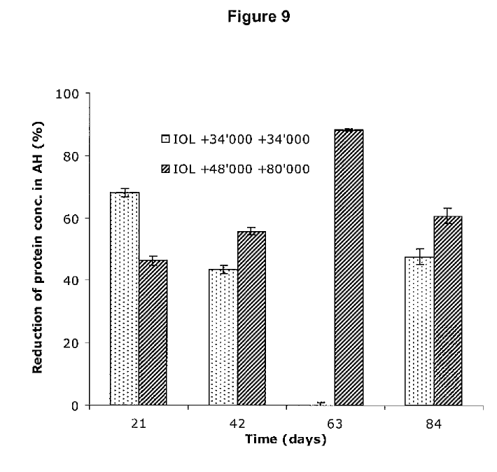

FIG. 9: Long term reduction of inflammation as measured by the protein concentration within the aqueous humor (AH). The value is expressed as a percentage in comparison to the control (insertion of IOL without DDS). Insertion of either IOL+two 34'0000DS or IOL+one 48'000 DDS and one 80'000 DDS.

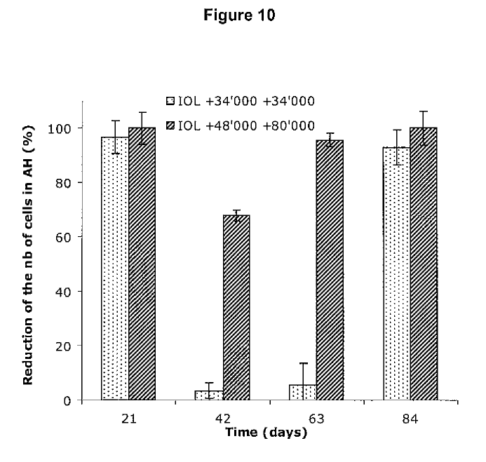

FIG. 10: Long term reduction of inflammation as measured by the number of inflammatory cells within the aqueous humor (AH). Same legend as in previous figure.

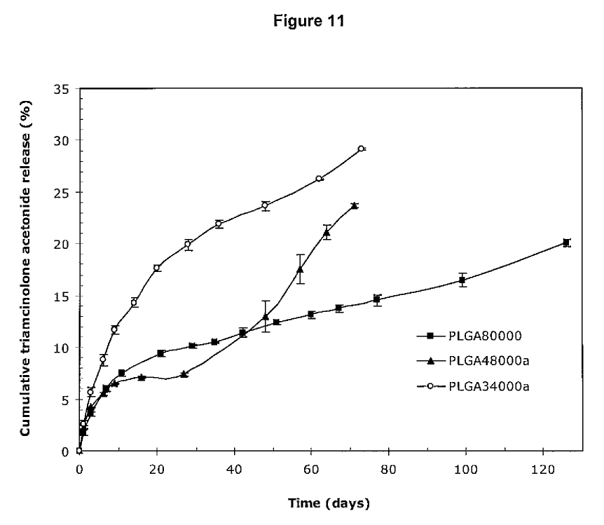

FIG. 11: Effects of the Mw of PLGA on in the vitro release from DDS at constant TA loading of 35%. ∘ DDS PLGA34000a, ▴DDS PLGA48000a, ▪ DDS PLGA80000. The values are means±SD (n=6).

The following examples illustrate the invention but should not be construed as being limiting.

### EXAMPLE 1

### Materials and Methods

### Materials

D,L-lactide-co-glycolide copolymer 50:50 lactide/glycolide with a weight-average molecular weight of 34 000 Da (Resomer RG503) and 48 000 Da (Resomer RG504) (abbreviated hereafter as PLGA-34000, and PLGA-48000, respectively) were purchased from Boehringer Ingelheim (Ingelheim, Germany). Intraocular lens (IOL) (AcrySof® MA 30BA) were supplied by Alcon Pharmaceuticals (Hunenberg, Switzerland). Triamcinolone acetonide (TA) was purchased from Sigma (Buchs, Switzerland); all reagents were of analytical grade.

### Preparation of Implants

The drug delivery systems/implants were prepared by dissolving 667 mg of polymer in 2 g of acetone. After complete dissolution of PLGA in acetone, TA was homogenously dispersed in polymer solution by vortexing for 5 min. DDS were prepared with poly(D,L-lactide-co-glycolide) (PLGA, Resomer®) with a MW of either 34'000 (RG 503) or 48'000 (RG 504). Unloaded DDS did not contain any active agent, loaded DDS contained about 35% triamcinolone acetonide (TA), corresponding to around 1000 μg of TA per disc.

Films were cast by pouring this dispersion into circular Teflon molds (35 mm in diameter), and allowing them to dry at room temperature during 3 days to evaporate the solvent. Thereafter the films were separated from the molds and stamped out to obtain discs. The films were shaped into discs having a mean diameter of 2 mm. A small perforation was made through the centre of the disc to allow the insertion of the haptic of the IOL (AcrySof® MA 30BA, Alcon Pharmaceuticals, Hünenberg, Switzerland). One or two discs were placed on the haptic. The implants combined with the IOL were sterilised by ethylene oxide gas sterilization.

### Assay of the Drug Content

Samples of discs were placed in 25.0 ml of acetonitrile to dissolve the PLGA and the TA. The amount of TA was measured by HPLC (Waters LC Module I plus, Milford, Mass., USA) using a C-18 reverse-phase column (Nucléosil C-18, 254 mm×4 mm×5 μm, Macherey Nagel, Switzerland) at a flow rate of 0.8 ml/min and was detected with a UV spectrophotometer at 236 nm. The mobile phase was composed of a mixture of water (60%) and acetonitrile (40%). Analysis was performed at 20° C. Under these experimental conditions, retention time of TA was 9.8 min.

### In Vitro Release Study

The implants were incubated in 5 ml of phosphate-buffered solution (0.1 M, pH 7.4) in a closed vial under smooth shaking at 37° C. At predetermined intervals, the entire buffer volume was sampled and 5 ml of fresh medium was added to the sample vial. The amount of TA released into the medium was measured by HPLC. Analysis of TA was conducted after filtration (Durapore, 0.2 μm, Mililipore Switzerland).

### Determination of TA in Explanted Implants

The recovered implants were dissolved in 25 ml of acetonitrile. The amount of TA remaining in the implants was determined by HPLC. In vivo release data were determined by measuring TA in recovered implants that had been placed for various periods of time.

The in vivo release of TA from the intraocular implant was calculated by the following equation: (the actual loading amount of TA in the implant before implantation)−(the remaining amount of TA in the recovered implant)/(the actual loading amount of TA in the implant before implantation)×100.

### Cataract Surgery and Implantation

The experiments and surgery were done on pigmented rabbits in accordance with the Swiss regulations for animal experimentation. Anaesthesia was performed by an i.m. injection of 1 ml/kg body weight of a mix (1:3) Rompun® 2% (Bayer, Lyssach, Switzerland): Ketalar® 50 mg/ml (Pfizer, Parke-Davis, Zürich, Switzerland). Mydriasis was induced by one drop of Tropicamide 0.5% SDU Faure (CIBA Vision, Hettlingen, Switzerland), Atropine 1% SDU Faure (Novartis Ophthalmics, Hettlingen, Switzerland) and Néosynéphrine 5% Faure (Europhta, Monaco). Both eyes were observed by slit lamp. Cataract surgery was performed on the right eye of pigmented rabbits, the contra-lateral eye being the control. After disinfections with aqueous Betadine® (Mundipharma, Hamilton, Bermuda), Oxybuprocaine 0.4% SDU Faure (Novartis) was instilled into the eye to obtain topical anesthesia. During surgery, the eye was constantly moistured with drops of Balanced Salt Solution (BSS®, Alcon Laboratories, Inc., Fort Worth, Tex., USA). Phacoemulsification was performed by the Series 20000™ Legacy® (Alcon Surgical, Fort Worth, Tex., USA). After ultrasonication and aspiration of the lens, the IOL+DDS was inserted. The wounds were sutured with Dafilon 10/0 (BiBraub, Aesculap AG, Tuttlingen, Germany). Okacin® (CIBA Vision, Hettlingen, Switzerland), an antibiotic, was instilled in the eye at the end of surgery and during the 3 following days.

The control group of rabbits (n=2) were inserted with IOL without DDS. The second group of rabbits was implanted with IOL wearing one unloaded disc of PLGA which did not contain any active agent (n=4). The third group (n=7) received an IOL wearing one disc (PLGA48000) loaded with TA (FIG. 4). The 4^{th} group received 2 discs (PLGA48000) loaded with TA for 42 days (n=2) (FIGS. 1 and 4). 5^{th} group: IOL wearing 2 discs loaded with TA, one PLGA48000 and one PLGA80000 (n=2). 6^{th} group: IOL wearing 2 discs PLGA34000 (n=2). In all experiments, the left, contralateral eye of each rabbit was used as a control.

### Evaluation of Inflammation

After implantation, rabbits were clinically followed at d 1, 2, 7, 14, 21 and 42 days. Corneal edema, conjunctival chemosis, conjunctival hyperemia and secretion were scored according to a semi-quantitative scale from 0 to 3. The clinical score was the sum of the 4 score parameters. The value 0 represented no symptoms/signs and 12 was the maximum. Intraocular pressure (IOP) was measured at d 0, 7, 21 and 42 d by a Tono-Pen® XL Applanation Tonometer (Medtronic Ophthalmics).

Punctures of aqueous humor (AH) were done under both systemic and topical anesthesia from both eyes at d 7, 14, 21, and 42 d. Protein concentration and number of inflammatory cells are low in normal calm eyes and increase during inflammation. Duplicate samples of AH were dried on slides for inflammatory cell counting after trypan blue staining. Remaining AHs were spun and acellular AH frozen down. Protein concentration was measured by the Coomassie® Plus Protein Assay Reagent Kit (Pierce, Rockford, Ill., USA)²¹. Results are given in means±SEM.

At the end of the experiment, systemic anesthesia, clinical evaluation, IOP measurement and final AH punctures were performed. Rabbits were euthanized by i.v. injection of pentobarbital. Both eyes were dissected into: conjunctiva, cornea, iris, lens (left eye) or IOL (right eye), vitreous humor, retina, choroid, sclera. Blood was withdrawn for serum preparation. All tissues were frozen away for further analysis.

### Measurement of TA Concentrations

Concentrations of TA have been determined by reversed phase HPLC using an LCI Module Plus instrument (Waters, Switzerland). For details, see section “Assay of the drug content” above.

### EXAMPLE 2

### Preparation of the Drug Delivery System

Four films were prepared to obtain the discs. All films appeared quite elastic and flexible and the discs were easily shaped from the initial films. Tables 1 and 2 summarize the results of the disc weight, thickness diameter and assay of TA content in each disc. The mean weight of the disc was 2.9 mg and the mean amount of TA per disc was 1021 μg corresponding to a constant TA loading of 35%.

TABLE 1
Characterization of the drug delivery system

| | | | | | Average |
|---|---|---|---|---|---|
| | | | | | amount |
| | | Average | Average | Average | of TA per |
| Batch | | weight | thickness | diameter | disc |
| # | Polymer | (mg)^{a} | (mm)^{a} | (mm)^{a} | (μg)^{a} |
| A | PLGA-34 000 | 2.90 ± 0.06 | 0.3 | 2.1 | 1040 ± 4 |
| B | PLGA-48 000 | 2.94 ± 0.11 | 0.3 | 2.1 | 1021 ± 47 |
| C | PLGA-48 000 | 2.96 ± 0.04 | 0.3 | 2.0 | 1003 ± 19 |
| D^{b} | PLGA-48 000 | 2.16 ± 0.06 | 0.2 | 2.0 | — |

TABLE 2
Characterization of further drug delivery systems

| | | | | | Average |
|---|---|---|---|---|---|
| | | | | | amount |
| | | Average | Average | Average | of TA per |
| | | weight | thickness | diameter | DDS |
| Polymer | Batch | (mg)^{a} | (mm)^{a} | (mm)^{a} | (μg)^{a} |
| PLGA34000 | PLGA | 2.90 ± 0.06 | 0.3 | 2.1 | 1040 ± 4 |
| | 34000a | | | | |
| | PLGA | 2.97 ± 0.11 | 0.3 | 2.0 | 1042 ± 61 |
| | 34000b | | | | |
| PLGA48000 | PLGA | 2.94 ± 0.11 | 0.3 | 2.1 | 1021 ± 47 |
| | 48000a | | | | |
| | PLGA | 2.96 ± 0.04 | 0.3 | 2.0 | 1003 ± 19 |
| | 48000b | | | | |
| | PLGA | 3.12 ± 0.06 | 0.3 | 2.1 | 1158 ± 23 |
| | 48000c | | | | |
| | PLGA | 2.16 ± 0.06 | 0.2 | 2.0 | — |
| | 48000d^{b} | | | | |
| PLGA80000 | PLGA | 2.97 ± 0.16 | 0.3 | 2.1 | 1038 ± 22 |
| | 80000 | | | | |

^{a}Mean of six measurements
^{b}Discs without TA

One (batches A and B) or two discs (batch C) were placed on the haptic of IOL. The disc and the haptic could be moved relative to each other. A schematic representation of the IOL combined with drug delivery system (DDS) is shown in FIG. 1.

Discs without TA were also prepared (batch D) to test the tolerability of the polymeric implant in rabbit eye.

### EXAMPLE 3

### In Vitro Release of TA

The cumulative release of TA from the batches A and B composed of PLGA-34000 and PLGA 48000 respectively has beer plotted in FIG. 2. The two types of drug delivery systems (or drug delivery devices) have different TA release profile due to the different molecular weight of the polymer. Data for discs B show a tri-phasic release profile, with an initial burst, a second stage followed by a second burst¹⁶. The initial burst (6%) may result from the rapid release of the drug adsorbed on the surface of the discs. This rapid release occurred within one week. During the first 24 hours, 28 μg of TA was released. After the initial burst, TA drug was released slowly during 3 weeks (diffusion phase). Swelling and disintegration of the polymer matrix are responsible of the second burst occurred after 4 weeks of incubation.

In the case of implants PLGA-34000 (batch A), no second burst was observed¹⁷, This may be due to the faster degradation rate of PLGA-34000 that allows the formation of water channels connected to the surface to the inside of the implant. Thus, TA can be released continuously by diffusion throughout the water channels, and degradation of the implant might proceed homogeneously.

Although implants PLGA-34000 showed a more rapid release at the beginning of the incubation, at the end of the in vitro assay period the total amount of drug released was almost the same as the amount released from the implants PLGA-48000. In fact, 282 μg of TA (29%) was released from implants PLGA-34000 after 73 days and 285 μg of TA (24%) from implants PLGA-48000 after 71 days.

The DDS from the batch PLGA80000 showed an initial burst (FIG. 11). This rapid release was not followed by a latency period as noticed for the disc PLGA48000a. TA release was gradual and very slow. After 18 weeks (126 d) of in vitro investigations, 20% of TA were released from the implants PLGA80000 corresponding to 216 μg of TA.

### EXAMPLE 4

### In Vivo Drug-Release Study

In vivo release was determined by measuring the remaining drug in recovered implants versus the initial content in the implant. FIG. 3 shows the profile of in vitro and in vivo TA release from the intraocular implants (PLGA48000b).

In contrast with the in vitro release profile, no initial burst was observed in vivo. After one week, the in vivo implants released hardly 1% of TA, whereas the in vitro TA-release was higher (6%). After 3 weeks, a mean of 78 μg (7 to 8%) of TA was released from the in vivo implants, approximately the same amount was released from the in vitro implants. Thereafter, the in vivo implants released the drug faster than it did in vitro, in fact, after 6 weeks, the mean amount released was 410 μg (41%) while in vitro was 116 μg (12%). The in vivo release rate is higher than the in vitro release rate, probably due to the increased TA solubility and to the increased degradation of the polymer in vivo. At the early stage, water absorption of the implant may be less than in vitro, resulting in no rapid release of the drug from the implant. In this condition, the water channels may not be well developed in the matrix. Finally, the drug may release rapidly after establishment of communication between inside and outside.

To increase the total amount of TA present in the delivery system, two discs (PLGA48000) were added to the haptic of each IOL (for details see Table 3). These IOL were implanted in rabbit eye (n=2) and the remaining drug in recovered implants was determined after 42 days. 39% of the initial amount of TA was released after 6 weeks from the two discs, which corresponds to 766 μg of TA. These results confirmed that the drug delivery system could release higher amount of TA in vivo than in vitro after 6 weeks.

TABLE 3
Total amount of TA released from the DDS during the in
vivo investigation

| | | | TA released | | Time |
|---|---|---|---|---|---|
| Rabbit # | Batch | DDS | % | (μg) | (week) |
| 40 | PLGA48000b | LB061E | 1.2 | 12 | 1 |
| 39 | | LB061D | 0 | 0 | |
| 42 | | LB061F | 7.4 | 74 | 3 |
| 37 | | LB061A | 8.1 | 80 | |
| 38 | | LB061C | 8.0 | 81 | |
| 45 | | LB064H | 55 | 564 | 6 |
| 46 | | LB064G | 26 | 256 | |
| 43 | | LB064CD* | 31 | 608 | |
| 44 | | LB064AB* | 46 | 914 | |
| 51 | PLGA34000b | LB063AB* | 61 | 779 | 12 |
| 52 | | LB063AB* | 56 | 901 | |

*IOL + 2 DDS

In summary, DDS loaded with around 1 mg of TA significantly reduced inflammation in the first days following surgery. Two DDS with a total load of 2 mg TA were more efficient than one DDS, since postoperative inflammation is totally inhibited during the first 3 weeks.

The same investigation was carried out with the batch PLGA34000b using two DDS per IOL. In this case also the total amount released of TA after 12 weeks was higher than in vitro. An average of 59% of the initial amount of TA was released in vivo, corresponding to 840 μg of TA (Table 3), whereas only 29% were released in vitro after 73 days (FIG. 11). Altogether, these results confirmed that the drug delivery system could release higher amount of TA in vivo than in vitro.

### EXAMPLE 5

### Effect of DDS on Inflammation

Cataract surgery and IOL insertion induced an inflammation. More than 10 mg/ml protein was measured in the aqueous humor (AH) (FIG. 5) and around 20 cells/microl AH was counted at d7 after surgery (FIG. 6). Both parameters progressively returned to normal values within around 4 weeks. The clinical score (FIG. 7) reached a value of 2-3, 7 days after cataract surgery. Then the value progressively decreased with an interval of time of 4 weeks.

Unloaded disc inserted with the IOL did not induce more inflammation than IOL without DDS. Values of protein concentrations and number of inflammatory cells within AH were comparable to those obtained after simple cataract surgery (FIG. 5A+B, FIG. 6A+B). Insertion of unloaded DDS did not worsen the clinical score either (FIG. 7). These results suggest that the DDS is well tolerated and is biocompatible.

Discs loaded with TA significantly reduced protein concentration and cell number within AH as compared to IOL without DDS, at time points d 7, d 14 and d 21 (FIG. 5A+B, FIG. 6A+B). One disc delivering TA allowed the eye to return to a calm situation 2-3 weeks after cataract surgery. The presence of 2 DDS loaded with TA totally suppressed inflammatory proteins exudation within AH, as shown at d 7 after surgery already (FIG. 5). The number of cells in AH was also reduced (FIG. 6). Insertion of 2 loaded DDS considerably reduced the clinical score which returned to null at d 14 after cataract surgery (FIG. 7). These observations prove that TA is delivered from the DDS within the first days after surgery and that 2 discs are more efficient than one disc in reducing inflammation. At d 42 after insertion of one loaded DDS, inflammation seems to be contained according to one parameter, the cell number in AH (FIG. 6A). But according to the parameter of protein concentration, some inflammation seems to reappear (FIG. 5A). This inflammation is inhibited by insertion of two loaded DDS (FIG. 5A).

In all experiments, left non-implanted eyes did not show protein exudation within AH, at any time and with any type of implant in the right eye (FIG. 5A). The clinical score of this eye stays also at zero (FIG. 7).

Intraocular pressure (IOP) mean varied between 7 and 17 mmHg over the time of the experiments, without any significant difference between implanted and control eyes (FIG. 8). The DDS loaded with TA did not induce pathological changes in IOP.

Increase of IOP is one of the usual side-effects of TA, after intraocular injections^{22,23}. Our study shows that IOP of the implanted eye of the rabbits is comparable to IOP in control eye. The absence of IOP elevation is probably due to the very low concentrations of the steroid in the eye. Indeed, 2 DDS contained 2 mg of TA whose only a small portion is delivered into the anterior chamber. For instance, at day 21, a total of 160 μg TA has been released into the eye.

Experiments were conducted over longer periods of time (84 d) to see whether 2 DDS could reduce long term inflammation. They were compared to cataract surgery and insertion of IOL without any DDS. The device comprised of one disc PLGA48000+one disc PLGA80000 was more efficient than the device comprised of one PLGA34000+one PLGA34000 in inhibiting inflammation. The PLGA48000+80000 device significantly inhibited protein concentration in AH by 46%, 56%, 88% and 61% at d 21, 42, 63 and 84, respectively (FIG. 9). Inhibition of inflammatory cells within AH reached 100%, 68%, 96% and 100% at d 21, 42, 63 and 84, respectively (FIG. 10).

### FURTHER REFERENCES

1. Hankinson, S.-E. Epidemiology of age-related cataract. In Principles and Practice of Ophtamlology; Albert, D.-M., Jakobiec, F.-A., Azar, D.-T., Gragoudas, E.-S., Eds.; W.B. Saunders Company: Philadelphia, 2000; pp. 511-521.
2. Simone, J. N.; Whitacre, M. M. Effects of anti-inflammatory drugs following cataract extraction. Curr. Opin. Opthalmol 2001, 12, 63-67.
3. Nagpal, M.; Nagpal, K.; Nagpal, P. N. Postcataract cystoid macular edema. Opthalmol Clin. North Am. 2001, 14, 651-9, ix.
4. Maurice, D. M.; Mishima, S. Ocular pharmacokinetics. In Pharmacology of the Eye; Sears, M. L., Ed.; Springer-Verlag: New York, 1984; pp. 19-116.
5. Park, C. H.; Jaffe, G. J.; Fekrat, S. Intravitreal triamcinolone acetonide in eyes with cystoid macular edema associated with central retinal vein occlusion. Am. J. Opthalmol 2003, 136, 419-425.
6. Parke, D. W. Intravitreal triamcinolone and endophthalmitis. Am. J. Opthalmol 2003, 136, 918-919.
7. Kunou, N.; Ogura, Y.; Yasukawa, T.; Kimura, H.; Miyamoto, H.; Honda, Y.; Ikada, Y. Long-term sustained release of ganciclovir from biodegradable scleral implant for the treatment of cytomegalovirus retinitis. J. Control Release 2000, 68, 263-271.
8. Yasukawa, T.; Kimura, H.; Tabata, Y.; Ogura, Y. Biodegradable scleral plugs for vitreoretinal drug delivery. Adv. Drug Deliv. Rev. 2001, 52, 25-36.
9. Okabe, K.; Kimura, H.; Okabe, J.; Kato, A.; Kunou, N.; Ogura, Y. Intraocular tissue distribution of betamethasone after intrascleral administration using a non-biodegradable sustained drug delivery device. Invest Opthalmol Vis. Sci. 2003, 44, 2702-2707.
10. Anand, R.; Font, R. L.; Fish, R. H.; Nightingale, S. D. Pathology of cytomegalovirus retinitis treated with sustained release intravitreal ganciclovir. Opthalmology 1993, 100, 1032-1039.
11. Chang, D. F.; Garcia, I. H.; Hunkeler, J. D.; Minas, T. Phase II results of an intraocular steroid delivery system for cataract surgery. Opthalmology 1999, 106, 1172-1177.
12. McColgin, A. Z.; Heier, J. S. Control of intraocular inflammation associated with cataract surgery. Curr. Opin. Opthalmol 2000, 11, 3-6.
13. Lingua, R. W.; Parel, J. M.; Assis, L. Absorbable copolymer clip: a potential substitute for sutures in strabismus surgery. Binoc. Vision 1987, 2, 129-136.
14. Lewis, D. H. Controlled release of bloactive agents from lactide/glycolide polymers. In Biodegradable Polymers As Drug Delivery System; Chasin, H., Langer, R., Eds.; Marcel Dekker: New York, 1990; pp. 1-43.
15. Holy, C. E.; Davies, J. E.; Shoichet, M. S. Bone tissue engineering on biodegradable polymers: preparation of a novel poly(lactide-co-glycolide) foam. In Biometarial, Carriers for Drug Delivery, and Scaffolds for Tissue Engineering.; Peppas, N. A., Mooney, D. J., Mikos, A. G., Brannon-Peppas, L., Eds.; A.I.Ch.E: New York, 1997; pp. 272-274.
16. Tojo, K.; Isowaki, A. Pharmacokinetic model for in vivo/in vitro correlation of intravitreal drug delivery. Adv. Drug Deliv. Rev. 2001, 52, 17-24.
17. Hashizoe, M.; Ogura, Y.; Kimura, H.; Moritera, T.; Honda, Y.; Kyo, M.; Hyon, S.-H.; Ikada, Y. Scleral plug of biodegradable polymers for controlled drug release in the vitreous. Arch. Opthalmol 1994, 112, 1380-1384.
18. Adamis A P, Shima D T. The role of vascular endothelial growth factor in ocular health and disease. Retina. 2005; 25:111-8.
19. Eyetech Study Group. Anti-vascular endothelial growth factor therapy for subfoveal choroidal neovascularization secondary to age-related macular degeneration: phase II study results. Opthalmology 2003; 110:979-86.
20. Tan D T, Chee S P, Lim L, Theng J, Van Ede M Randomized clinical trial of Surodex steroid drug delivery system for cataract surgery: anterior versus posterior placement of two Surodex in the eye. Opthalmology 2001; 108:2172-81.
21. Bradford M M. A rapid and sensitive method for the quantitation of microgram quantities of protein utilizing the principle of protein-dye binding. Anal. Biochem. 1976; 72:248-54.
22. Ciulla T A, Walker J D, Fong D S, Criswell M H. Corticosteroids in posterior segment disease: an update on new delivery systems and new indications. Curr. Opin. Ophtha/moL 2004; 15:211-20.
23. Jonas J B, Degenring R F, Kreissig I, Akkoyun I, Kamppeter B A. Intraocular pressure elevation after intravitreal triamcinolone acetonide injection. Opthalmology. 2005; 112:593-8.
24. E I Harazi S M, Feldman R M. Control of intra-ocular inflammation associated with cataract surgery. Curr. Opin. Opthalmol. 2001; 12:4-8.
25. Taylor H R, Keeffe J E. World blindness: a 21st century perspective. Br. J. Opthalmol. 2001; 85:261-6.
26. Guttman C. VEGF inhibitor effective in early AMD trials. ESCRS Eurotimes 2004; 9:1-3.
27. Ambati J, Ambati B K, Yoo S H, Ianchulev S, Adamis A P. Age-related macular degeneration: etiology, pathogenesis, and therapeutic strategies. Surv. Opthalmol. 2003; 48:257-93
28. Yasukawa T. Ogura Y, Sakurai E, Tabata Y, Kimura H. Intraocular sustained drug delivery using implantable polymeric devices. Adv. Drug Deliv. Rev. 2005; 57:2033-46.
29. Adamis, A P, et al. Arch. Opthalmol. 1996; 114: 66-71.
30. The Eyetech study group. Retina 2002; 22: 143-152.
31. Krzystolik, A G, et al. Arch. Opthalmol. 2002; 120: 338-346.
32. Iturralde, D. Retina 2006; 26: 270-284.
33. Bhistikul, R B. et al. Arch. Opthalmol. 2005; 123: 214-219.
34. Evereklioglu, C et al. Surv. Opthalmol. 2005; 50: 297-350.

1-21. (canceled)

22. A device comprising an intraocular lens and a drug delivery system attached thereto, wherein said drug delivery system is or can be loaded with one or more therapeutic agents, and wherein said drug delivery system is attached to a haptic of said intraocular lens.

23. A device comprising an intraocular lens and one or more drug delivery systems attached thereto, wherein said drug delivery systems are or can be loaded with one or more therapeutic agents, and wherein said drug delivery system(s) is/are attached to (a) haptic(s) of said intraocular lens.

24. The device of claim 23, wherein more than one drug delivery system is attached to said intraocular lens, and wherein different drug delivery systems are or can be loaded with different therapeutic agents.

25. The device of any one of claims 22 to 24, wherein said drug delivery system comprises a biodegradable matrix material.

26. The device of claim 25, wherein said biodegradable matrix material is selected from the group consisting of: poly(lactic acid), poly(glycolic acid), poly(lactide-co-glycolide), polycaprolactones, polyorthoesters, polyanhydrides, polyesteramides and mixtures thereof.

27. The device of any one of claims 22 to 24, wherein said drug delivery system comprises a non-biodegradable matrix material.

28. The device of claim 27, wherein said non-biodegradable matrix material is selected from the group consisting of: methacrylates, acrylates and co-polymers thereof, silicone, and polymers and co-polymers of vinyl acetate, vinyl pyrrolidone and vinyl alcohol.

29. The device of claim 22, wherein the release of said therapeutic agent(s) from said drug delivery system occurs within a period of tip to one year.

30. The device of claim 22, wherein the shape of said drug delivery system is selected from: disc, cylinder, nail-like-plug, rod, tablet, sphere, truncated cone with a bended axis, polymer wire rolled around a haptic, and polymer sheet wrapped around a haptic.

31. The device of claim 22, wherein said therapeutic agent(s) is/are selected from anti-inflammatory agents, anti-vasoproliferative agents, immunosuppressive agents, antibiotics, antiviral agents, anti-mitotic agents and combinations thereof.

32. The device of claim 31, wherein said anti-inflammatory agents are selected from the group consisting of corticosteroids, cycloplegics and non-steroidal anti-inflammatory drugs.

33. The device of claim 22, wherein said therapeutic agent(s) is/are selected from one or more of the following: triamcinolone acetonide, betamethasone, dexamethasone, prednisolone, fluorometholone, anti-VEGF agents, anti-TNF agents, gaciclovir, acyclovir and foscavir.

34. The device of claim 22, wherein said therapeutic agent(s) is/are selected from the group consisting of: antisense oligonucleotides, aptamers, antibodies and fragments thereof, peptides and constructs for gene therapy.

35. A method of treatment and/or prevention of one or more diseases or malfunctions of the eye, comprising implanting a device of claim 22 into an eye of a patient.

36. A method of treatment and/or prevention of one or more diseases or malfunctions of the eye, comprising
(a) manufacturing a device comprising an intraocular lens and one or more drug delivery systems; and
(b) implanting said device into an eye of a patient,
wherein said manufacturing comprises attaching said drug delivery system(s) to (a) haptic(s) of said intraocular lens, and wherein said drug delivery system(s) is/are or can be loaded with one or more therapeutic agents.

37. The method of claim 35 or 36, wherein said disease or malfunction is selected from the group consisting of: a condition requiring implantation of an intraocular lens, a condition arising from or aggravated by the implantation of an intraocular lens, a condition present before implantation, and combinations thereof.

38. The method of claim 37, wherein said condition requiring implantation of an intraocular lens is cataract or is expected to result in cataract.

39. The method of claim 37, wherein said condition arising from or aggravated by the implantation of an intraocular lens is selected from: postoperative inflammation, capsule opacification (secondary cataract), uveitis, cystoid macular edema (CME) and retinopathies.

40. The method of claim 35, wherein said disease or malfunction is selected from the group consisting of: uveitis; age-related macular degeneration (AMD); retinopathies including diabetic retinopathy, retinitis including retinitis pigmentosa, viral retinitis such as cytomegalovirus (CMV) retinitis, and proliferative vitreoretinopathy; endophthalmitis and choroidal neovascularization.
